(19) United States
(12) Patent Application Publication
(10) Pub. No.: US 2026/0053394 A1
(43) Pub. Date: Feb. 26, 2026

(54) SYSTEM FOR DETECTING AND MONITORING GLUCOSE LEVELS AND METHOD THEREOF

(71) Applicant: GLUTRACK HEALTHCARE PRIVATE LIMITED, Maharashra (IN)

(72) Inventors: Anil Vishwanath SONKAR, Maharashtra (IN); Anil Yuvaraj KOTNIS, Maharashtra (IN)

(21) Appl. No.: 19/104,212

(22) PCT Filed: Aug. 23, 2023
(86) PCT No.: PCT/IN2023/050794

§ 371 Date: Feb. 15, 2025

(30) Foreign Application Priority Data
Aug. 23, 2022 (IN) 2022210480

(51) Int. Cl. A61B 5/145; A61B 5/00; A61B 5/01; A61B 5/05; A61B 5/1455; A61B 5/1495

(52) U.S. Cl. CPC A61B 5/14532; A61B 5/01; A61B 5/05; A61B 5/14517; A61B 5/14539; A61B 5/14551; A61B 5/1495; A61B 5/7282; A61B 5/742; A61B 5/746; A61B 2560/0223; A61B 2560/0468; A61B 2562/0233; A61B 2562/0247; A61B 2562/0271

(57) ABSTRACT

The present subject matter discloses system (100) and method (700) for detecting and monitoring glucose levels. The system (100) comprising wearable device (200) and wearable device (200) comprising a plurality of transceivers (202) configured for transmitting plurality of power packets to one or more transceivers (202) from plurality of transceivers (202) thereby pushing each of, electrolytes under skin (500) of a user, glucose transmitters and glucose molecules towards surface of the skin (500). Plurality of electrodes (204) configured for collecting ions from body section towards the plurality of electrodes (204). Spectroscopic laser (206) and a spectroscopic detector (208) configured for detecting glucose molecules pushed towards surface of the body section. Processor (210) configured for monitoring the glucose levels according to the detection of the glucose levels.

### CROSS-REFERENCE TO RELATED APPLICATIONS AND PRIORITY

The present application claims priority from Indian patent application number 202221048080 filed on Aug. 23, 2022.

### TECHNICAL FIELD

The present subject matter described herein, in general, relates to a field of electronic devices, and more particularly to system and method for detecting and monitoring glucose levels.

### BACKGROUND

Unlike conventional times, medical facilities have been upgraded in terms of smart equipment, devices and infrastructure. With proliferation digital equipment, easy to handle devices have replaced other complex and bulky devices.

In the today's hi-tech world, human beings are more encountered by health issues and chronic conditions. The most widely chronic condition is fluctuations in glucose levels, where a person's body may see elevated blood glucose levels that result from defects in the body's ability to produce and/or use insulin. There are three main types of diabetes are defined in a scientific way. Type 1 of the known three types diabetes is mainly caused by an autoimmune reaction (the body attacks itself by mistake) that stops body from making insulin. Type 1 diabetes occurs in which the body doesn't use insulin well and can't keep the blood sugar or glucose at normal levels. Gestational diabetes, which develops in pregnant women who have never had diabetes and usually resolves spontaneously after delivery.

Further, fluctuations in the blood glucose levels are also sensitive to exercise, sleep, stress, smoking, travel, illness, menses, and other psychological and lifestyle factors unique to individuals. To have a healthier life it is very necessary to identify the likely occurrence of the chronic condition such as diabetes.

Out of many known and widely adopted ways, commonly available solutions require chemicals or insertion of electrodes on to the skin or may also requires sticking tattoos or any other chemical or enzymatically treated material on the skin surface for measuring glucose. Such techniques are not reliable and painful to the individuals. Moreover, these conventional techniques may lead to infections in the skin and might also be time consuming, inaccurate and complex in nature.

### SUMMARY

Before the present system for detecting and monitoring glucose levels and method thereof are described, it is to be understood that this application is not limited to the particular systems, and methodologies described, as there can be multiple possible embodiments which are not expressly illustrated in the present disclosure. It is also to be understood that the terminology used in the description is for the purpose of describing the particular versions or embodiments only, and is not intended to limit the scope of the present application. This summary is provided to introduce concepts related to the system for detecting and monitoring glucose levels and the method thereof are further described below in the detailed description. This summary is not intended to identify essential features of the claimed subject matter nor is it intended for use in determining or limiting the scope of the claimed subject matter.

In one implementation, a system for detecting and monitoring glucose levels is disclosed. The system comprises a wearable device and the wearable device comprises a plurality of transceivers configured for transmitting a plurality of power packets to one or more transceivers from the plurality of transceivers thereby pushing each of, electrolytes under skin of a user, glucose transmitters and glucose molecules towards surface of the skin. The wearable device further comprises a plurality of electrodes configured for collecting ions from the body section towards the plurality of electrodes. The wearable device further comprises a spectroscopic laser and a spectroscopic detector configured for detecting glucose molecules pushed towards surface of the body section. The wearable device further comprises a processor configured for monitoring the glucose levels according to the detection of the glucose levels.

In another implementation, a method for detecting and monitoring glucose levels is disclosed. The method comprising a wearable device configured for transmitting, through a plurality of transceivers, a plurality of power packets to one or more transceivers from the plurality of transceivers thereby pushing each of, electrolytes under skin of a user, glucose transmitters and glucose molecules towards surface of the skin. The method further may comprises creating, a plurality of electrodes configured for collecting ions from the body section towards the plurality of electrodes. The method further may comprises detecting, through a spectroscopic laser and a spectroscopic detector, glucose molecules pushed towards surface of the body section. The method further may comprises monitoring, through a processor, the glucose levels according to the detection of the glucose levels.

### BRIEF DESCRIPTION OF THE DRAWINGS

The foregoing detailed description of embodiments is better understood when read in conjunction with the appended drawings. For the purpose of illustrating the disclosure, example constructions of the disclosure are shown in the present document; however, the disclosure is not limited to the specific methods and systems disclosed in the document and the drawings.

The detailed description is given with reference to the accompanying figures. In the figures, the left-most digit(s) of a reference number identifies the figure in which the reference number first appears. The same numbers are used throughout the drawings to refer like features and components.

FIG. 1 illustrates a network implementation of a system for detecting and monitoring glucose levels, in accordance with an embodiment of the present subject matter;

FIG. 2 illustrates a method for detecting and monitoring glucose levels, in accordance with an embodiment of the present subject matter; and

FIG. 3 illustrates an exemplary wearable device, in accordance with an embodiment of the present subject matter.

### DETAILED DESCRIPTION

Some embodiments of this disclosure, illustrating all its features, will now be discussed in detail. The words “comprising,” “having,” “containing,” and “including,” and otherforms thereof, are intended to be equivalent in meaning and be open ended in that an item or items following any one of these words is not meant to be an exhaustive listing of such item oritems, or meant to be limited to only the listed item or items. It must also be noted that as usedherein and in the appended claims, the singular forms “a,” “an,” and “the” include plural references unless the context clearly dictates otherwise. Although any systems and methods similar or equivalent to those described herein can be used in the practice or testing of embodiments of the present disclosure, the exemplary, system for detecting and monitoring glucose levels and method thereof are now described. The disclosed embodiments are merely exemplary of the disclosure, which may be embodied in various forms.

Various modifications to the embodiment will be readily apparent to those skilled in the art and the generic principles herein may be applied to other embodiments. However, one of ordinary skill in the art will readily recognize that the present disclosure for the system for detecting and monitoring glucose levels and the method thereof is not intended to be limited to the embodiments illustrated, but is to be accorded the widest scope consistent with the principles and features described herein.

As referred in the background, existing methods require chemicals or insertion of electrodes on to the skin and requires sticking tattoos or any other chemical or enzymatically treated material on the skin surface for measuring glucose. Such techniques are not reliable and painful to the individuals. Moreover, those may lead to infections in the skin, time consuming, inaccurate and complex in nature.

The present subject matter overcomes the limitations associated with the existing methods.

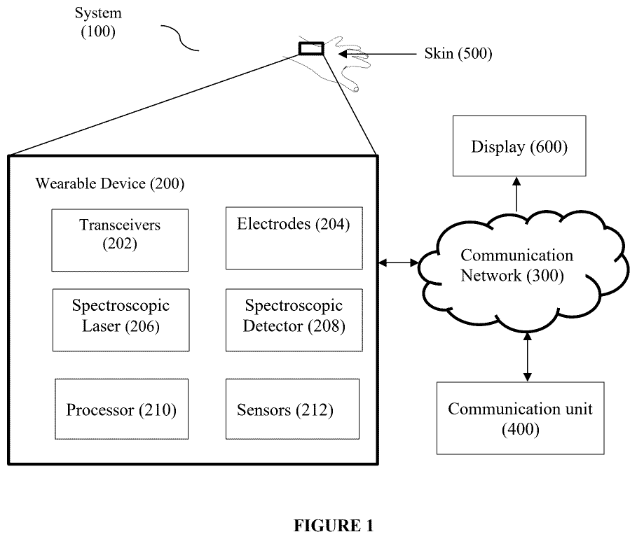

Referring now to FIG. 1, in an exemplary embodiment, a network implementation of a system (100) for detecting and monitoring glucose levels is disclosed. The present subject matter is explained considering that, a wearable device (200) may be considered as a portable electronic device, or a benchtop device or a combination of the benchtop device and the wearable device (200). Further, it will be understood that the communication unit (400) is electronic devices or applications residing on the electronic device. Examples of the communication unit (400) may include, but are not limited to, a portable computer, a personal digital assistant, a handheld device, and a workstation. The communication unit (400) may be communicatively coupled to the wearable device (200) through a communication network (300).

In one implementation, the communication network (300) may be a wireless network, a wired network or a combination thereof. The communication network (300) may be implemented as one of the different types of networks, such as intranet, local area network (LAN), wide area network (WAN), theinternet, and the like. The communication network (300) may either be a dedicated network or a shared network. The shared network represents an association of the different types of networks that use a variety of protocols, for example, Hypertext Transfer Protocol (HTTP), Transmission ControlProtocol/Internet Protocol (TCP/IP), Wireless Application Protocol (WAP), Over the Air (OTA), Bluetooth, Infra-red and the like, to communicate with one another. Further, the communication network (300) may include a variety of network devices, including routers, bridges, servers, computing devices, storage devices, cloud storage and the like.

In one embodiment, the system (100) comprises of the wearable device (200). The wearable device (200) comprises a plurality of transceivers (202) configured for transmitting a plurality of power packets to one or more transceivers (202) from the plurality of transceivers (202). The transceivers are coils that create a magnetic field across a cross section of a body part (for example wrist) and thus pushes each of, electrolytes under skin (500) of a user, glucose transmitters and glucose molecules towards surface of the skin (500).

The system (100) comprises of a plurality of electrodes (204) configured anywhere on the wearable device (200) for collecting ions from a body section towards the plurality of electrodes (204). Further, a spectroscopic laser (206) and a spectroscopic detector (208) are configured for detecting glucose molecules pushed towards surface of the body section or boy part. Further, a processor (210) is configured for monitoring the glucose levels according to the detection of the glucose levels. The glucose levels include a low glucose level or a high glucose level.

The system (100) further comprises a communication unit (400) integrated within the wearable device (200) for receiving data about the glucose levels generated after the monitoring. The system (100) further comprises a display (600) for displaying the data (glucose levels) to the user. The data is displayed in form of a report in a preselected format.

The system (100) is also configured for generating alerts in case the glucose levels detected shows one of the low glucose level or the high glucose level. low glucose level and the high glucose level may be identified according to a threshold present in the system (100) and may also be customized by the user. For example, glucose level is detected as the low glucose level when the glucose value is lower than 65 mg/dL and the glucose level is detected as the high glucose level when the glucose value is higher than 200 mg/dL. The system (100) may also generate an average of the glucose levels for a predefined time (set by the user for example, 3 months). The alert may be sent through the communication unit (400) over the display (600). The alert may be in the audio-visual form. Further, the system (100) comprises plurality of sensors (212) to detect various biological parameters. The biological parameters comprise temperature, oxygen level, pressure and pH at the surface of the body. The biological parameters may be used for showing a variation the glucose levels.

For example, by measuring the temperature from biological parameters, an effect of the temperature over the glucose levels is considered. With the change in the temperature at the surface of the body, the water distribution also changes. Due to change in water distribution, the glucose level may also change. Such change in the glucose levels is also monitored and measured by the system (100). Therefore, system (100) monitors and considers any change in the biological parameter to further determine the change in the glucose levels.

In another example, the pH may also increase or decrease at the surface of the body according to change in the water distribution of the body. Monitoring the pH of the skin may help in detecting any major deviations in the glucose levels and such monitoring through the system (100) may help in controlling the glucose levels by the user.

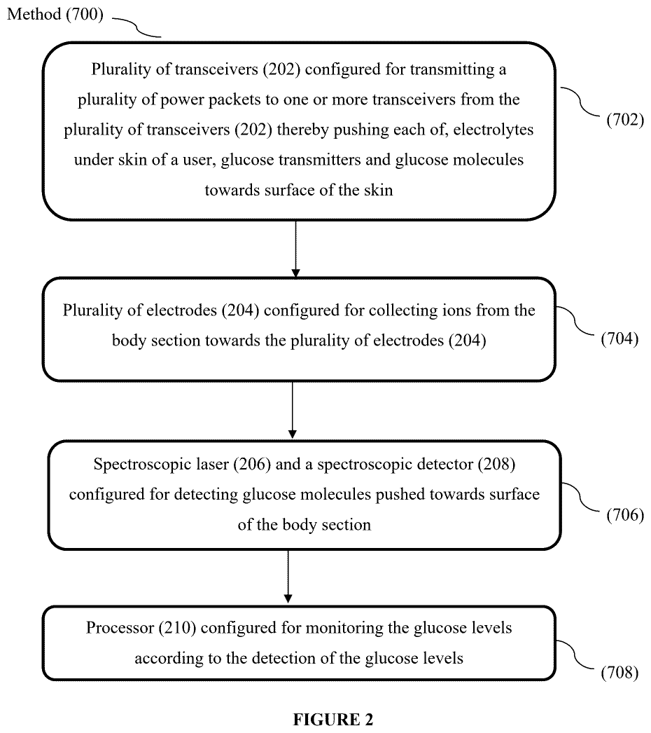

In another embodiment, referring to the FIG. 2, method (700) for detecting and monitoring glucose levels is disclosed. The method may be executed by the system (100).
At step 702: The plurality of transceivers (202) transmits a plurality of power packets to one or more transceivers (202) from the plurality of transceivers (202) thereby pushing each of, electrolytes under skin (500) surface of a user, glucose transmitters and glucose molecules towards surface of the skin.
At step 704: The plurality of electrodes (204) are configured for collecting ions from the body section towards the plurality of electrodes (204).
At step 706: The spectroscopic laser (206) and a spectroscopic detector (208) are configured for detecting glucose molecules pushed towards the surface of the body section.
At step 708: The processor (210) is configured for monitoring the glucose levels according to the detection of the glucose levels.

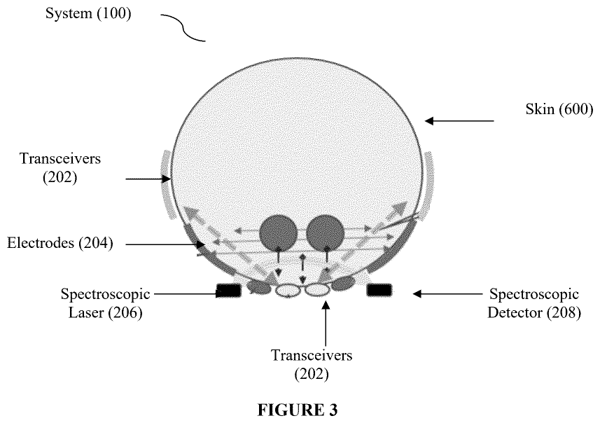

Now again referring to the FIG. 1 in combination with FIG. 3, FIG. 3 discloses an exemplary wearable device (200). The electrodes (204) are placed on or around the body section for collecting the electrolyte and transporting ions towards the skin (500) surface.

The body section comprises blood vessels, vein or artery. In the system (100), the spectroscopic laser (206) and the spectroscopic detector (208) are configured to detect glucose from the blood and the sweat.

In the system (100), the transceivers (202) may comprises wireless transceivers. A part of the wearable device (200) may be worn around the body section and with a close contact with the skin (500).

In the system (100), the processor (210) is configured for performing a self-calibration and a cross calibration while monitoring the glucose levels from the blood and sweat. The self-calibration and the cross calibration comprises identifying variations between detected glucose in the sweat and the blood based on a comparison of the detected glucose levels with threshold glucose values.

Further, The self-calibration and the cross calibration comprises tracking the variations between the detected glucose in the sweat and the blood. Further, The self-calibration and the cross calibration comprises analyzing the variations in the detected glucose for removing errors in calibrations or readings during sudden increases or decrease of the blood glucose in the body.

As an exemplary embodiment, the variations between glucose identified in the sweat, and the blood may be tracked and analyzed to remove the skews in the calibrations and/or readings during sudden increases or decrease of the glucose levels in the body.

As an exemplary embodiment the spectroscopic laser (206) and the spectroscopic detector (208) uses spectroscopic techniques for detecting the glucose levels. The spectroscopic techniques comprises infrared spectroscopy, optical spectroscopy, fluorescence spectroscopy, Ultra Violet (UV) spectroscopy, circular dichroism spectroscopy.

The system (100) comprises of a plurality of sensors (212). As an example the sensors (212) are temperature sensors, SpO₂ (Peripheral Capillary Oxygen Saturation) sensors, pH sensor and pressure sensors. The temperature sensors are used for measuring a temperature of the body. The SpO₂ sensors are used for measuring an oxygen level of the body. The pressure sensor used for measuring the body pressure.

In an exemplary embodiment, the sensors (212) are used in the system (100) for activity detection of the body. As per the activity detected in the body, the change in the glucose level occurs and the pH of skin (500) may also change which may be identified by the pH sensor. The activity may comprise running, sleeping, walking, jumping or any other physical activity. In the system (100) the activities and the corresponding variations of the biological parameters i.e., temperature, oxygen level, pressure and pH are stored and used by the processor (210) during monitoring of the glucose levels.

Exemplary embodiments discussed above may provide certain advantages. Though not required to practice aspects of the disclosure, these advantages may include those provided by the following features.

Some embodiments of the system (100) and method (700) performs non-invasive glucose detection and continuous monitoring of glucose levels.

Some embodiments of the system (100) and method (700) detects hypoglycemia and hyperglycemia for healthy individuals and for the patients suffering from type 1 and type 2 diabetes.

Some embodiments of the system (100) and method (700) eliminates the use of chemicals or insertion of electrodes on to the skin.

Some embodiments of the system (100) and method (700) eliminates the need to stick tattoos or any other chemical or enzymatically treated material on the skin (500) surface.

Although implementations for system (100) and method (700) for monitoring blood glucose level have been described, it is to be understood that the appended claims are not necessarily limited to the specific features or methods described. Rather, the specific features and methods are disclosed as examples of implementations for monitoring blood glucose level in the body.

1. A system (100) for detecting and monitoring glucose levels, the system (100) comprising:
a wearable device (102), comprising:
a plurality of transceivers (202) configured for transmitting a plurality of power packets to one or more transceivers (202) from the plurality of transceivers (202) thereby pushing each of, electrolytes under skin of a user, glucose transmitters and glucose molecules towards surface of the skin (500);
a plurality of electrodes (204) configured for collecting ions from a body section towards the plurality of electrodes (204);
a spectroscopic laser (206) and a spectroscopic detector (208) configured for detecting glucose molecules pushed towards surface of the body section; and
a processor (210) configured for monitoring the glucose levels according to the detection of the glucose levels.

2. The system (100) as claimed in the claim 1, wherein the body section comprises blood vessels, vein or artery.

3. The system (100) as claimed in the claim 1, wherein the spectroscopic laser (206) and the spectroscopic detector (208) are configured to detect glucose from the blood and the sweat.

4. The system (100) as claimed in the claim 1, wherein the transceivers (202) comprises wireless transceivers.

5. The system (100) as claimed in the claim 1, wherein the processor (210) is configured for:
performing a self-calibration and the cross calibration while monitoring the glucose levels from the blood and sweat, wherein the self-calibration and the cross calibration comprises:
identifying variations between detected glucose in the sweat and the blood based on a comparison of the detected glucose levels with threshold glucose values;
tracking the variations between the detected glucose in the sweat and the blood; and
analysing the variations in the detected glucose for removing errors in calibrations or readings during sudden increases or decrease of the blood glucose in the body.

6. The system (100) as claimed in claim 1, wherein the a spectroscopic laser (206) and a spectroscopic detector (208) uses spectroscopic techniques for detecting the glucose levels, wherein the spectroscopic techniques comprises infrared spectroscopy, optical spectroscopy, fluorescence spectroscopy, Ultra Violet (UV) spectroscopy, circular dichroism spectroscopy.

7. The system (100) as claimed in claim comprises:
a communication unit (300) integrated within the wearable device (200) for receiving data about the glucose levels generated after the monitoring; and
a display (600) for displaying the data, wherein the data is displayed in form of a report in a preselected format.

8. The system (100) as claimed in the claim 1, wherein the wearable device (200) comprising:
temperature sensors, SpO₂ (Peripheral Capillary Oxygen Saturation) sensors, pH sensor pressure sensors for measuring a temperature of the body and oxygen level of the body.

9. The system (100) as claimed in the claim 1, wherein the processor is configured to:
identify in case the glucose levels are higher or lower then threshold values; and
generate alerts when the glucose levels are identified as one of the higher glucose levels or lower glucose levels.

10. A method (700) for detecting and monitoring glucose levels, the method (700) comprising a wearable device configured for:
transmitting, through a plurality of transceivers (202), a plurality of power packets to one or more transceivers (202) from the plurality of transceivers (202) thereby pushing each of, electrolytes under skin of a user, glucose transmitters and glucose molecules towards surface of the skin;
creating, a plurality of electrodes (204) configured for collecting ions from the body section towards the plurality of electrodes (204);
detecting, through a spectroscopic laser (206) and a spectroscopic detector

(208) glucose molecules pushed towards surface of the body section; and
monitoring, through a processor (210), the glucose levels according to the detection of the glucose levels.
